(19) United States
(12) Patent Application Publication
(10) Pub. No.: US 2017/0319440 A1
(43) Pub. Date: Nov. 9, 2017

(54) COSMETIC COMPOSITION CARRIER COMPRISING LATEX FOAM

(71) Applicant: AMOREPACIFIC CORPORATION, Seoul (KR)

(72) Inventors: Jung Sun CHOI, Yongin-si (KR); Kyung Ho CHOI, Yongin-si (KR); Yeong Jin CHOI, Yongin-si (KR)

(21) Appl. No.: 15/520,033

(22) PCT Filed: Oct. 22, 2015
(86) PCT No.: PCT/KR2015/011242

§ 371 Date: Apr. 18, 2017

(30) Foreign Application Priority Data
Oct. 22, 2014 (KR) 10-2014-0143473

(51) Int. Cl. A61K 8/02; A61K 8/06; A61K 8/37; A45D 34/00; C08J 9/00; A61Q 17/04; A61K 8/34; A45D 40/22

(52) U.S. Cl. CPC A61K 8/0208; A61Q 17/04; A61K 8/062; A61K 8/34; A61K 8/37; A45D 34/00; C08J 9/0061; A45D 40/22; A61K 2800/87; C08J 2205/044; C08J 2309/08; C08J 2407/02; C08J 2409/10

(57) ABSTRACT

The present invention provides a cosmetic composition carrier comprising a latex foam having a density of 0.05-0.2 g/cm³ and a cosmetic product comprising same. The cosmetic composition carrier according to the present invention has excellent filling, holding, and discharge capabilities with respect to a liquid cosmetic, has excellent antibacterial activity, can be filled with, hold, and discharge liquid cosmetic compositions in a wide range of low to high viscosities, thus allows excellent color expression and uniform application without caking when a liquid cosmetic composition held in the cosmetic composition carrier is applied to the skin, and allows the application of the liquid cosmetic composition to a portable container.

### TECHNICAL FIELD

The present disclosure relates to a cosmetic composition carrier including a latex foam.

### BACKGROUND ART

Recently, there has been an increasing need for carrying out or correcting make-up with ease even at the outdoor, and thus an easily portable carrier for a liquid cosmetic composition has been required increasingly. Containers that allow high portability of a liquid cosmetic composition may include a pact type container. In order for a pact type container to receive a liquid cosmetic composition, it is required to consider whether or not a cosmetic composition carrier can be used for a pact type container, whether or not a cosmetic composition can be packed well in the carrier, whether or not the carrier can support the cosmetic composition homogeneously for a long time, and whether or not an adequate amount of cosmetic composition can be discharged when the cosmetic composition is to be taken out of the carrier. In addition, such a carrier is exposed continuously to the exterior during use and is in contact with the skin through an applicator, and thus preferably is made of a material strongly resistant against bacterial contamination.

In the case of a conventional latex foam, it has a small pore size and thus disadvantageously provides a small vacant space in which the contents can be supported. A latex that has been used as an applicator to date is problematic in that it has no supportability and dischargeability suitable for a pact-type formulation, when using a high-viscosity cosmetic composition is used.

### DISCLOSURE

### Technical Problem

A technical problem to be solved by the present disclosure is to provide a cosmetic composition carrier which has excellent packability, supportability and dischargeability with respect to liquid cosmetic agents having different viscosities, shows antibacterial activity, and discharges a liquid cosmetic agent supported therein dividedly to provide an excellent cosmetic effect in terms of color expression and uniform application.

### Technical Solution

In one general aspect, there is provided a cosmetic composition carrier which includes a latex foam having a density of 0.05-0.2 g/cm³.

According to an embodiment, the latex foam may include pores whose average pore size of 200-800 μm.

According to another embodiment, the latex foam may have a thickness of 1-50 mm.

According to still another embodiment, the latex foam may have a hardness of 30-95 as determined by a durometer before a cosmetic composition is supported therein.

According to still another embodiment, the latex may be at least one selected from the group consisting of butadiene rubber (BR), styrene-butadiene rubber (SBR), natural rubber (NR), chloroprene rubber (CR), butyl rubber (isoprene-isobutylene rubber: IIR), isoprene rubber (IR), vulcanized ethylene-propylene rubber (EPR), polysulfide rubber, silicone rubber, fluororubber, urethane rubber, acrylic rubber, ethylene propylene diene monomer (EPDM) rubber, polyvinyl alcohol (PVA) and ethylene vinyl acetate (EVA).

According to still another embodiment, the cosmetic composition carrier may be for use in taking and applying the cosmetic composition supported therein with an applicator.

According to still another embodiment, the latex foam may have a coating film having closed cells on at least one of the bottom surface opposite to the surface in contact with an applicator and lateral surfaces.

According to still another embodiment, the latex foam may have a cell-in-cell structure including smaller cells in larger cells.

According to yet another embodiment, the latex foam may have antibacterial activity.

In another aspect, there is provided a cosmetic product which includes: the cosmetic composition carrier including a latex foam; and a cosmetic composition supported in the carrier.

According to an embodiment, the cosmetic product may further include an applicator with which the cosmetic composition is taken out of the latex foam.

According to another embodiment, the cosmetic composition may have a viscosity of 3,000-30,000 cps.

According to still another embodiment, the cosmetic composition may include an alcohol.

According to still another embodiment, the cosmetic composition may have a formulation selected from the group consisting of a solution, emulsion, gel, cream and suspension.

According to yet another embodiment, the cosmetic composition may be selected from the group consisting of a skin care cosmetic composition, makeup cosmetic composition, hair care cosmetic composition and a UV protecting agent.

### Advantageous Effects

According to the embodiments of the present disclosure, the cosmetic composition carrier has excellent packability, supportability and dischargeability with respect to a liquid cosmetic agent, shows excellent antibacterial activity, and allows packing, support and discharge of liquid cosmetic compositions having a broad range of viscosities from a low viscosity to a high viscosity. Therefore, when applying the liquid cosmetic composition supported in the cosmetic composition carrier to the skin, it is possible to realize excellent color expression and to accomplish uniform application without caking. In addition, it is possible to apply a liquid cosmetic composition to an easily portable container.

### BEST MODE

Exemplary embodiments now will be described more fully hereinafter. This disclosure may, however, be embodied in many different forms and should not be construed as limited to the exemplary embodiments set forth therein.

As used herein, “carrier” means a material by which any material or ingredient, such as a composition, can be supported, and is used interchangeably with “support”, “impregnation material” or “medium”. For example, the composition supported in the carrier may be delivered to the skin through an applicator (also referred to as an application means) or application tool), including a hand, puff, tip, brush, or the like.

As used herein, “foam” means one formed by foaming a polymer through a dry or wet foaming process.

As used herein, “supportability” or “supporting capability” means ability of holding and retaining any material or ingredient. The supportability required for a carrier is different from taking a material transiently on an applicator in that the supportability relates to supporting a composition uniformly for a long time.

As used herein, “packability” or “packing capability” means ability of packing a cosmetic composition in foam and may be expressed by the time required for packing a predetermined amount of cosmetic composition in foam. Herein, measurement of “packability” or “packing capability” relates to the time required for packing a cosmetic composition having a viscosity of 3,000-30,000 cps manually in a latex foam having a size of circle diameter 44 mm×height 9.0 mm.

As used herein, “dischargeability” or “discharging capability” means the amount of cosmetic composition discharged when taking the cosmetic composition by an applicator from the foam in which the cosmetic composition is supported. It is preferred for the foam to discharge the cosmetic composition in an adequate amount neither too little nor too much. Herein, measurement of “dischargeability” or “discharging capability” relates to the value obtained when compressing the surface of a latex foam including a cosmetic composition supported therein and having a size of circle diameter 44 mm×height 9.0 mm under a weight (force) of 2 kg.

As used herein, “durability” means how long the foam having a cosmetic composition supported therein maintains its shape without melting, tearing or swelling, when the foam is allowed to stand at a predetermined temperature for a predetermined time, and/or how much the foam resist against repeated pressure applied by an applicator, when the cosmetic composition is taken by the applicator from the foam.

In one aspect, there is provided a cosmetic composition carrier which includes a latex foam having a density of 0.05-0.2 g/cm³. More particularly, the latex foam may have a density of 0.07-0.2 g/cm³.

Herein, the latex foam may also be referred to as foamed latex. The latex foam has a polymerized polymer and pores having air. Herein, the density of a latex foam means the weight of a latex component based on the total volume of the latex in the cosmetic composition carrier. According to some embodiments of the present disclosure, the cosmetic composition carrier may include a latex in an amount of 80 wt % or more, 85 wt % or more, 90 wt % or more, 93 wt % or more, 95 wt % or more, 97 wt % or more, 98 wt % or more, 99 wt % or more, or 100 wt %, based on the total weight of the foam, but is not limited thereto.

According to some embodiments of the present disclosure, it is possible for the latex foam to have pores in which a cosmetic composition may be supported by controlling the density thereof. According to an embodiment, when the latex foam has a density of 0.05-0.2 g/cm³, it is possible for the latex to have a space sufficient for supporting a cosmetic composition therein and to provide excellent durability. When the latex foam has a density lower than 0.05 g/cm³, the weight of a latex component which is a solid content in the latex foam is too low to provide a sufficient degree of tensile strength, elasticity and hardness, thereby providing poor durability. When the latex foam has a density higher than 0.2 g/cm³, the weight of a latex component which is a solid content in the latex foam is too high and the proportion of the latex based on the total volume is high, thereby providing a small space occupied by air, i.e., a low porosity. Thus, in this case, the latex foam has a small volume in which the contents may be supported.

According to another embodiment, the pores contained in the latex foam may have a pore size of 200-800 μm, preferably 300-800 μm, and more particularly 400-700 μm. Particularly, the pore size may be 800 μm or less, 700 μm or less, 600 μm or less, 500 μm or less, 400 μm or less, 300 μm or less, or 200 μm, and 200 μm or more, 300 μm or more, 400 μm or more, 500 μm or more, 600 μm or more, 700 μm or more, or 800 μm.

Herein, the average pore size of the latex foam may be determined by using an optical microscope (Nikon ECLIPSE LV100POL) at a magnification of 5×/0.15.

When the pore size is less than 200 μm, the latex foam has a small pore space in which a cosmetic composition is supported, and thus shows poor packability. When the pore size is larger than 800 μm, the latex foam has a pore space which is too large to provide high dischargeability and supportability.

According to still another embodiment of the present disclosure, the latex foam may have a pore size controlled to 200-800 μm so that it may have high air permeability, cushioning effect, softness, flexibility and elasticity and provide improved packability and supportability. A larger pore size may cause degradation of divisibility upon discharge. To improve divided dischargeability, the latex foam may have a cell-in-cell structure. As used herein, “cell-in-cell structure” means a structure including smaller cells in larger cells. Particularly, smaller cells are contained in the backbone or framework (or stem) of cells. When the carrier is foamed to have a cell-in-cell structure, it is possible to take the contents with an applicator (such as puff) over a large area thereof without caking and to deliver the contents uniformly to the skin without caking. In addition, such a cell-in-cell foam structure causes no deposition of a cosmetic composition even when the cosmetic composition supported in the foam has a low viscosity. Thus, it is possible to realize excellent color expression and uniform application, thereby providing a high-quality cosmetic effect.

According to still another embodiment, the latex foam may have a thickness of 1-50 mm. Particularly, the thickness may be 50 mm or less, 40 mm or less, 30 mm or less, 20 mm or less, 10 mm or less, or 5 mm or less, and 1 mm or more, 10 mm or more, 20 mm or more, 30 mm or more, 40 mm or more, or 50 mm. When the latex foam has a thickness less than 1 mm, the amount of a cosmetic composition supported therein becomes too small. When the latex foam has a thickness larger than 50 mm, it is difficult to discharge the contents (cosmetic composition) without any residue thereof during use. Particularly, the latex foam carrier may have a thickness of 3-45 mm, preferably 5-40 mm, more preferably 8-35 mm, and most preferably 10-30 mm.

According to still another embodiment, the latex foam may have a hardness of 30-95 as determined by a durometer before a cosmetic composition is supported therein. For example, the hardness determined by a durometer may be Asker F hardness.

As used herein, “Asker F hardness” means hardness as determined by ASKER DUROMETER Type F (a hardness measuring system available from ASKER), and refers to hardness before a cosmetic composition is supported in foam.

When the latex foam has a hardness less than 30, it is too soft to support a cosmetic composition with a uniform distribution and it may discharge a cosmetic composition excessively. When the latex foam has a hardness larger than 95, it is too hard to pack a cosmetic composition therein and has a difficulty in discharging a cosmetic composition. In this context, the latex foam according to an embodiment of the present disclosure preferably has an Asker F hardness of 30-90, more preferably 30-80 and even more preferably 40-70. Particularly, the latex may have an Asker F hardness of 95 or less, 90 or less, 80 or less, 70 or less, 60 or less, 50 or less, 40 or less, or 30, and 30 or more, 40 or more, 50 or more, 60 or more, 70 or more, 80 or more, 90 or more, or 95.

According to still another embodiment, the latex may be at least one selected from the group consisting of butadiene rubber (BR), styrene-butadiene rubber (SBR), natural rubber (NR), chloroprene rubber (CR), butyl rubber (isoprene-isobutylene rubber: IIR), isoprene rubber (IR), vulcanized ethylene-propylene rubber (EPR), polysulfide rubber, silicone rubber, fluororubber, urethane rubber, acrylic rubber, ethylene propylene diene monomer (EPDM) rubber, polyvinyl alcohol (PVA) and ethylene vinyl acetate (EVA). For example, the latex foam may include styrene-butadiene rubber (SBR), and at least one of butyl rubber (isoprene-isobutylene rubber: IIR) and isoprene rubber (IR).

According to still another embodiment, as the latex supports the contents (cosmetic composition) therein, the vacant space in the carrier is occupied by the contents (cosmetic composition) and volumetric swelling may occur. The degree of such swelling may be varied with materials, hardness and additives and may be calculated according to the following formula:

$$Height swelling ratio ( \% ) = ( Height of carrier after supporting contents - Original height of carrier ) \text{/} Original height of carrier \times 100 \\ Diameter swelling ratio ( \% ) = ( Diameter of carrier after supporting contents - Original diameter of carrier ) \text{/} Original diameter of carrier \times 100$$

According to still another embodiment, the latex foam shows a height swelling ratio of 1-20% and a diameter swelling ratio of 1-30%, after supporting a cosmetic composition therein. Therefore, the latex foam may be processed to have a size smaller than the final size by 1-20% in height and by 1-30% in diameter, considering such swelling ratios.

The above-described latex foam having a density of 0.05-0.2 g/cm³ and including pores having an average pore size of 200-800 μm shows excellent packability and supportability with respect to a cosmetic composition and also provides excellent dischargeability. In addition, when the latex foam has an Asker F hardness of 30-95, it provides better supportability, packability and dischargeability with respect of cosmetic compositions having a viscosity of 3,000-30,000 cps.

According to still another embodiment, the cosmetic composition carrier may be for use in taking and applying the cosmetic composition supported therein with an applicator. Herein, there is no particular limitation in the applicator, as long as it allows a cosmetic composition to be taken out of a cosmetic composition carrier (latex foam) in which the cosmetic composition is supported. For example, the applicator may include a hand, puff, tip or brush and may also be referred to as an application means. In other words, the latex foam according to the present disclosure is not an applicator but a carrier, and thus a separate applicator should be used to take the cosmetic composition out of the latex foam.

In general, a liquid cosmetic composition is more difficult to carry and store as compared to a solid cosmetic composition. However, when using the cosmetic compositions carrier according to an embodiment of the present disclosure, it is possible to carry and store a liquid or creamy cosmetic composition stably and safely. In addition, when a liquid or creamy cosmetic composition is supported in the carrier according to still another embodiment, it is possible to reduce a momentary change in physical properties caused by thermal or physical impact. When taking a cosmetic composition with an applicator and using it, the carrier functions to fix the cosmetic composition so that only an adequate amount of cosmetic composition may be paid off.

Further, the latex foam according to an embodiment of the present disclosure has antibacterial activity, retains such antibacterial activity against the external bacteria and provides an excellent antibacterial effect.

The cosmetic composition carrier according to an embodiment of the present disclosure may have at least one of the following characteristics:

i) a supportability of 13.5-14.8 g, as determined by measuring the amount of a cosmetic agent held in the carrier in a container after 15 g of the cosmetic composition is packed in the carrier and is allowed to stand for 4 weeks;

ii) a packability of 15-25 seconds, as determined by measuring the time required for packing 15 g of a cosmetic composition;

iii) a dischargeability of 0.3-0.5 g, as determined by measuring the pay-off amount of a cosmetic composition when carrying out pay-off of the cosmetic composition from the carrier with an applicator;

iv) an antibacterial activity of 99% or more, as determined by the test method according to KSK0693;

v) a height swelling ratio of 1-20% after supporting a cosmetic composition; and

vi) a diameter swelling ratio of 1-30% after supporting a cosmetic composition.

According to still another embodiment, the antibacterial activity may be determined by using the test method according to KSK0693.

According to still another embodiment, the latex foam may have a coating film having closed cells on at least one of the surface (also referred to as ‘bottom surface’ hereinafter) opposite to the surface in contact with an applicator and lateral surfaces. By virtue of such a coating film, it is possible to prevent a cosmetic composition from being leaked through the bottom surface or lateral surfaces while the cosmetic composition is supported in the carrier. When the carrier has a coating film type surface on the bottom surface or lateral surfaces thereof as mentioned above, it is possible to inhibit the cosmetic composition from moving toward the lateral surfaces or bottom surface during use and from being leaked out of the carrier, and thus to reduce a loss of the cosmetic composition. As a result, it is possible to increase the service life and improve the convenience.

In another aspect, there is provided a cosmetic product including: the cosmetic composition carrier including a latex foam; and a cosmetic composition supported in the carrier. The cosmetic product includes a cosmetic composition applied to the latex foam according to an embodiment of the present disclosure. Thus, the cosmetic product allows well packing of the cosmetic composition, provides excellent supportability, allows discharge of an adequate amount of the cosmetic composition when taking the cosmetic composition, maintains excellent durability for a long time, and shows an antibacterial effect.

The cosmetic product according to an embodiment of the present disclosure may further include an applicator with which the cosmetic composition is taken out of the latex foam carrier. The applicator is the same as described above.

According to an embodiment, the cosmetic composition may include a liquid cosmetic composition having a viscosity ranging from a low viscosity to a high viscosity. There is no particular limitation in viscosity but the cosmetic composition may have a viscosity of 3,000-30,000 cps (centi poise). Particularly, the cosmetic composition may have a viscosity of 30,000 cps or less, 20,000 cps or less, 10,000 cps or less, 7,000 cps or less, or 3,000 cps or less, and 3,000 cps or more, 7,000 cps or more, 10,000 cps or more, 20,000 cps or more, or 30,000 cps.

When the cosmetic composition has a viscosity less than 3,000 cps, such a low viscosity of the composition may cause deposition or separation of contents. When the cosmetic composition has a viscosity larger than 30,000 cps, such a high viscosity may cause degradation of packability.

As used herein, the viscosity may be determined by using a viscometer. For example, the viscosity may be determined by using LVDV II+PRO with spindle No. 64 at a spindle speed of 5 rpm or 12 rpm, preferably with liner 3500-5000, spindle No. 63 at 12 rpm. In a variant, the viscosity may be determined by using BROOKFIELD RVDV-III ULTRA (Serial No. RY6521152), 64 spindle, 12 rpm. In the case of a liquid cosmetic composition, the viscosity may be determined by using Brookfield DV-III ULTRA with spindle No. 63 at a spindle speed of 5 rpm.

A viscosity of 3,000-30,000 cps of a liquid cosmetic composition may be determined at 30° C. for 60 seconds, but is not limited thereto.

There is no particular limitation in the cosmetic composition according to an embodiment of the present disclosure. However, the cosmetic composition may have a formulation selected from the group consisting of a solution, emulsion, gel, cream and suspension.

According to another embodiment, the cosmetic composition may be an emulsion or dispersion, and particularly may be an oil-in-water (O/W) type, water-in-oil (W/O) type, oil dispersed or water dispersed formulation.

According to still another embodiment, the cosmetic composition may include an alcohol. Particularly, the alcohol may include at least one selected from monohydric, dihydric and trihydric alcohols, and/or at least one selected from primary, secondary and tertiary alcohols.

The carrier according to an embodiment of the present disclosure causes little swelling even when it supports a cosmetic composition including an alcohol, and thus ensures the stability as a carrier. When applying such a cosmetic composition to the skin through an application means, it is possible to impart a cooling effect to the skin, thereby providing an excellent feeling of use.

According to still another embodiment, the cosmetic composition may be selected from the group consisting of a skin care cosmetic composition, makeup cosmetic composition, hair care cosmetic composition and a UV protecting agent. Particularly, the cosmetic composition may be formulated into make-up primer, make-up base, foundation, powder, twin cake, lipstick, lip gloss, eye shadow, eyebrow, concealer, lip liner, blusher, UV protecting agent, lotion, cream or essence, or the like, but is not limited thereto.

The cosmetic product according to an embodiment of the present may be provided in the form of a cosmetic container called generally ‘pact’, which includes a lower part capable of receiving a carrier for a cosmetic composition and an upper lid part to which a mirror may be attached, but is not limited thereto.

According to yet another embodiment, when the cosmetic composition is a UV protecting agent, it is possible to provide a cosmetic product with high stability and portability regardless of the viscosity of a UV protecting agent by impregnating the cosmetic composition carrier according to the present disclosure with the UV protecting agent. In addition, there is no need for washing hands separately after the UV protecting agent is taken out of the carrier and applied to the skin. Thus, it is possible to enjoy a continuous UV protecting effect with ease even in an outdoor environment.

### MODES FOR INVENTION

Exemplary embodiments now will be described more fully hereinafter. The present disclosure may, however, be embodied in many different forms and should not be construed as limited to the exemplary embodiments set forth therein.

### EXAMPLES

Styrene-butadiene rubber (SBR) latex is used as a main ingredient and a pigment and vulcanizing agent are compounded therewith in water optionally with a foaming agent and other additives. Then, the resultant mixture is agitated and foamed, injected to a mold and solidified, and heated in a heating furnace to carry out molding along with vulcanization. The resultant product is cut, dried and subjected to surface processing to obtain a styrene-butadiene rubber (SBR) foam.

The obtained styrene-butadiene rubber (SBR) foam has an Asker hardness of 40 and an average pore size of 680 μm.

### Test Example 1

1-1: Results of Supportability, Dischargeability and Packability Depending on Specific Gravity of Styrene-Butadiene Rubber (SBR) Foam

In this example, supportability, packability and dischargeability are determined depending on specific gravity of a styrene-butadiene rubber (SBR) foam.

The cosmetic composition having a viscosity of 15,000 cps and described in the following Table 1 is supported in each styrene-butadiene rubber (SBR) foam having a density of 0.01-0.25 g/cm³, Asker F hardness of 20-95 before supporting the cosmetic composition and having a size of diameter 44 mm×height 9.0 mm. Then, the supportability, dischargeability and packability of each foam are determined.

The packability is determined by the time where 15 g of the cosmetic composition is packed. The supportability is determined by the amount of the cosmetic composition retained in the foam, when 15 g of the cosmetic composition is packed and allowed to stand at 55° C. for 4 weeks. The dischargeability is determined by the amount of the cosmetic composition when the contents supported by each foam are paid off by using puff and hands. The results are shown in the following Table 2.

TABLE 1
Cosmetic Compositions Supported in Foam

| | | | Amount |
|---|---|---|---|
| | | Ingredients | (wt %) |
| Oil phase | Oily | Ozokerite | 2.0 |
| ingredients | ingredient | | |
| | Oily | Dicaprylyl carbonate | 10.00 |
| | ingredient | | |
| | Preservative | Methyl paraben | 0.100 |
| | UV | Octylmethoxy cinnamate | 7.000 |
| | protecting | | |
| | agent | | |
| | UV | Isoamyl-P-methoxy cinnamate | 2.000 |
| | protecting | | |
| | agent | | |
| | Pigment | Disteardimonium hectorite | 1.50 |
| | Oily | Decamethylcyclopentasiloxane | 16.00 |
| | ingredient | | |
| | Emulsifier | Sorbitan sesquioleate | 2.000 |
| | Emulsifier | Lauryl PEG.PPG-18.18methicone | 1.500 |
| | Pigment | Polymethyl methacrylate | 5.00 |
| | Pigment | Titanium dioxide/aluminum | 7.00 |
| | | hydroxide/stearic acid | |
| Aqueous | | Water | To 100 |
| phase | | | |
| ingredients | | | |
| | Moisturizer | Glycerin | 8.000 |
| | Emulsion | Salt | 1.00 |
| | stabilizer | | |
| | | Fragrance | 0.400 |

| | |
|---|---|
| Total | 100.000 |

TABLE 2
Supportability, Dischargeability and Packability of Styrene-Butadiene
Rubber (SRB) Foam Depending on Density

| | Foam density before supporting (g/cm³) | | | | | |
|---|---|---|---|---|---|---|
| | 0.01 | 0.05 | 0.1 | 0.15 | 0.2 | 0.25 |
| Supportability (g) | 11.0 | 13.3 | 14.0 | 14.5 | 14.5 | 12.5 |
| Dischargeability (g) | 0.8 | 0.5 | 0.45 | 0.43 | 0.40 | 0.27 |
| Packability (sec) | 60 | 15 | 15 | 15 | 20 | 65 |

1-2: Analysis of Supportability, Packability and Dischargeability Depending on Hardness of Styrene-Butadiene Rubber (SRB) Foam

In this example, supportability, packability and dischargeability are determined depending on hardness of a styrene-butadiene rubber (SBR) foam.

TABLE 3
Supportability, Dischargeability and Packability of Styrene-Butadiene
Rubber (SRB) Foam Depending on Hardness

| | Foam hardness before supporting (Asker F) | | | | | | | | |
|---|---|---|---|---|---|---|---|---|---|
| | 20 | 30 | 40 | 50 | 60 | 70 | 80 | 90 | 95 |
| Sup- | 13.0 | 13.7 | 13.7 | 14.0 | 14.0 | 14.3 | 14.3 | 14.3 | 13.3 |
| port- | | | | | | | | | |
| ability | | | | | | | | | |
| (g) | | | | | | | | | |
| Dis- | 0.7 | 0.4 | 0.4 | 0.45 | 0.45 | 0.45 | 0.45 | 0.4 | 0.27 |
| charge- | | | | | | | | | |
| ability | | | | | | | | | |
| (g) | | | | | | | | | |
| Pack- | 45 | 20 | 20 | 20 | 15 | 15 | 15 | 20 | 35 |
| ability | | | | | | | | | |
| (sec) | | | | | | | | | |

As shown in Table 3, a styrene-butadiene rubber (SBR) foam having an Asker F hardness of 30-80 before supporting a cosmetic composition shows significantly better packability as compared to a foam having an Asker F hardness of 20. Such a foam also shows adequate dischargeability. It is preferred that a carrier shows a dischargeability corresponding to an amount neither too little nor too much. Thus, a dischargeability of about 0.3-0.5 g per pay-off is judged as adequate amount. When the dischargeability is less than 0.3 g, an excessively small amount of cosmetic agent is applied, resulting in degradation of a cosmetic effect. When the dischargeability is larger than 0.5 g, caking occurs, resulting in degradation of a uniform application effect. Although there is no significant difference in supportability, the styrene-butadiene rubber (SBR) foam having an Asker F hardness of 30-80 is shown to have better supportability.

1-3: Analysis of Supportability, Dischargeability and Packability Depending on Pore Size of Styrene-Butadiene Rubber (SBR) Foam

The cosmetic composition according to Table 1 and a styrene-butadiene rubber (SBR) foam having an average pore size of 200-800 μm and a size of diameter 44 mm×height 9.0 mm are used to determine the supportability, dischargeability and packability of each foam in the same manner as described in 1-1. The results are shown in Table 4.

TABLE 4
Supportability, Dischargeability and Packability of Styrene-Butadiene
Rubber (SRB) Foam Depending on Pore Size

| | Foam pore size (μm) | | | | | | | | |
|---|---|---|---|---|---|---|---|---|---|
| | 160 | 200 | 300 | 400 | 500 | 600 | 700 | 800 | 850 |
| Sup- | 13.2 | 14.3 | 14.5 | 14.5 | 14.3 | 14.0 | 14.0 | 13.5 | 12.9 |
| port- | | | | | | | | | |
| ability | | | | | | | | | |
| (g) | | | | | | | | | |
| Dis- | 0.22 | 0.3 | 0.3 | 0.37 | 0.4 | 0.43 | 0.45 | 0.47 | 0.6 |
| charge- | | | | | | | | | |
| ability | | | | | | | | | |
| (g) | | | | | | | | | |
| Pack- | 60 | 25 | 25 | 20 | 20 | 15 | 15 | 15 | 15 |
| ability | | | | | | | | | |
| (sec) | | | | | | | | | |

As shown in Table 4, when using a styrene-butadiene rubber (SBR) foam having a pore size of 200-800 μm as a carrier, it is possible to obtain adequate dischargeability and high packability. In addition, it is shown that a styrene-butadiene rubber (SBR) foam having a pore size of 200-600 μm has the highest supportability.

1-4: Analysis of Supportability and Dischargeability Depending on Viscosity of Cosmetic Composition

A styrene-butadiene rubber (SBR) foam having a pore size of 550 μm, an Asker F hardness of 50 before supporting a cosmetic composition and a size of diameter 44 mm×height 9.0 mm is prepared, and the foam is used to support each of the cosmetic compositions having a viscosity of 1,500-30,000 cps as shown in the following Table 5 therein. Then, supportability and dischargeability are determined in the same manner as described in 1-1. As shown in Table 6, when using a cosmetic composition having a viscosity of 2,000-30,000 cps, it is possible to obtain excellent supportability. In addition, an adequate range of dischargeability is obtained in the above-defined range.

TABLE 5
Cosmetic Compositions Having Different Viscosities

| Ingredients | | | | | | | | | |
|---|---|---|---|---|---|---|---|---|---|
| Amount (wt %) | #1 | #2 | #3 | #4 | #5 | #6 | #7 | #8 | #9 |
| Ozokerite | 12 | 10 | 6 | 5 | 5 | 4 | 1 | 1 | 1 |
| Dicaprylyl | 10 | 10 | 10 | 10 | 10 | 10 | 10 | 10 | 10 |
| carbonate | | | | | | | | | |
| Octylmethoxy | 7 | 7 | 7 | 7 | 7 | 7 | 7 | 7 | 7 |
| cinnamate | | | | | | | | | |
| Disteardimonium | 1.7 | 1.5 | 1.2 | 0.7 | 0.5 | 0.4 | 0.4 | 0.35 | 0.3 |
| hectorite | | | | | | | | | |
| Decamethyl | 16 | 16 | 16 | 16 | 16 | 16 | 16 | 16 | 16 |
| cyclopentasiloxane | | | | | | | | | |
| Sorbitan | 2 | 2 | 2 | 2 | 2 | 2 | 2 | 2 | 2 |
| sesquioleate | | | | | | | | | |
| Lauryl PEG. | 1.5 | 1.5 | 1.5 | 1.5 | 1.5 | 1.5 | 1.5 | 1.5 | 1.5 |
| PPG- | | | | | | | | | |
| 18.18methicone | | | | | | | | | |
| Polymethyl | 5 | 5 | 5 | 5 | 5 | 5 | 5 | 5 | 5 |
| methacrylate | | | | | | | | | |
| Titanium | 7 | 7 | 7 | 7 | 7 | 7 | 7 | 7 | 7 |
| dioxide/iron | | | | | | | | | |
| oxide | | | | | | | | | |
| Water | To 100 | To 100 | To 100 | To 100 | To 100 | To 100 | To 100 | To 100 | To 100 |
| Glycerin | 8 | 8 | 8 | 8 | 8 | 8 | 8 | 8 | 8 |
| Salt | 1 | 1 | 1 | 1 | 1 | 1 | 1 | 1 | 1 |
| Viscosity (cps) | 32,000 | 30,000 | 25,000 | 20,000 | 15,000 | 10,000 | 5,000 | 3,000 | 1,500 |

TABLE 6
Supportability, Dischargeability and Packability Depending on Viscosity
of Cosmetic Composition

| | Viscosity (cps) | | | | | | | | |
|---|---|---|---|---|---|---|---|---|---|
| | 1,500 | 3,000 | 5,000 | 10,000 | 15,000 | 20,000 | 25,000 | 30,000 | 32,000 |
| Packability | 35 | 15 | 15 | 18 | 18 | 23 | 23 | 23 | 45 |
| (sec) | | | | | | | | | |
| Supportability | 13.2 | 13.5 | 13.5 | 14.0 | 14.0 | 14.0 | 14.3 | 14.3 | 13.3 |
| (g) | | | | | | | | | |
| Dischargeability | 0.26 | 0.3 | 0.3 | 0.33 | 0.35 | 0.40 | 0.44 | 0.45 | 0.65 |
| (g) | | | | | | | | | |

1-5: Antibacterial Activity of Styrene-Butadiene Rubber (SBR) Foam

Antibacterial activity is determined in terms of a decreasing rate after the inoculation with Staphylococcus aureus and Klebsiella pneumonia. The antibacterial activity may be determined by using the test method according to KSK0693 and the strains used in this example are Staphylococcus aureus ATCC 6538 and Klebsiella pneumonia ATCC 4352. The results are shown in the following Table 7.

TABLE 7
Antibacterial Activity of Styrene-Butadiene Rubber (SBR) Foam

| | SBR Foam |
|---|---|
| Decreasing rate of Staphylococcus aureus | 99% |
| Decreasing rate of Klebsiella pneumonia | 99% |

### [Test Example 2] Evaluation of Swelling Ratio and Stability of Styrene-Butadiene Rubber (SBR) Foam

A polyurethane foam and SBR foam each having a size of diameter 44 mm×height 9.0 mm is prepared and each foam is impregnated with 15 g of 95% ethanol. Then, the swelling degree of each foam is determined.

TABLE 8

| | Styrene-Butadiene | | | |
|---|---|---|---|---|
| | Rubber (SBR) Foam | | Polyurethane Foam | |
| Hardness | 50 | | 70 | |
| Pore size (μm) | 550 | | 350 | |
| | Diameter % | Height % | Diameter % | Height % |
| Variation after | 2.31 | 0.77 | 12.65 | 16.41 |
| supporting ethanol | | | | |
| (95%) | | | | |

As shown in Table 8, as each foam absorbs ethanol, the polyurethane foam undergoes swelling to cause an increase in diameter and height. However, when ethanol is supported in the styrene-butadiene rubber (SBR) foam, no swelling of the foam appears.

In addition, as shown in the following Table 9, provided is a foam including a blend of styrene-butadiene rubber (SBR) with natural rubber (NR), isoprene rubber (IR) or butyl rubber (IIR) and having a pore size of 550 μm, Asker F hardness of 50 before supporting a cosmetic composition and a size of diameter 44 mm×height 9.0 mm. The foam is impregnated with 15 g of 95% ethanol and the swelling degree is determined.

TABLE 9

| | | Amount of styrene- | |
|---|---|---|---|
| | | butadiene rubber | |
| | Amount (%) | (SBR) (%) | Stability |
| Natural rubber (NR) | 10 | 90 | Swelled |
| Isoprene rubber (IR) | 10 | 90 | Stable |
| Butyl rubber (IIR) | 10 | 90 | Stable |

As shown in Table 9, the foam including a blend of styrene-butadiene rubber (SBR) with isoprene rubber (IR) or butyl rubber (IIR) causes swelling, while the foam including a blend of natural rubber (NR) with styrene-butadiene rubber (SBR) causes no swelling and is more stable structurally.

In brief, according to a first embodiment, there is provided a cosmetic composition carrier which may include a latex foam having a density of 0.05-0.2 g/cm³.

According to a second embodiment, there is provided a cosmetic composition carrier according to the first embodiment, wherein the latex foam may include pores having an average pore size of 200-800 μm.

According to a third embodiment, there is provided a cosmetic composition carrier according to the first or second embodiment, wherein the latex foam may have a thickness of 1-50 mm.

According to a fourth embodiment, there is provided a cosmetic composition carrier according to any one of the first embodiment to the third embodiment, wherein the latex foam may have a hardness of 30-95 as determined by using a durometer before a cosmetic composition is supported therein.

According to a fifth embodiment, there is provided a cosmetic composition carrier according to any one of the first embodiment to the fourth embodiment, wherein the latex may be at least one selected from the group consisting of butadiene rubber (BR), styrene-butadiene rubber (SBR), natural rubber (NR), chloroprene rubber (CR), butyl rubber (isoprene-isobutylene rubber: IIR), isoprene rubber (IR), vulcanized ethylene-propylene rubber (EPR), polysulfide rubber, silicone rubber, fluororubber, urethane rubber, acrylic rubber, ethylene propylene diene monomer (EPDM) rubber, polyvinyl alcohol (PVA) and ethylene vinyl acetate (EVA).

According to a sixth embodiment, there is provided a cosmetic composition carrier according to any one of the first embodiment to the fifth embodiment, wherein the latex foam may include styrene-butadiene rubber (SBR); and at least one of butyl rubber (isoprene-isobutylene rubber: IIR) and isoprene rubber (IR).

According to a seventh embodiment, there is provided a cosmetic composition carrier according to any one of the first embodiment to the sixth embodiment, which may be for use in taking and applying the cosmetic composition supported therein with an applicator.

According to an eighth embodiment, there is provided a cosmetic composition carrier according to any one of the first embodiment to the seventh embodiment, wherein the latex foam may have a coating film having closed cells on at least one of the bottom surface opposite to the surface in contact with an applicator and lateral surfaces.

According to a ninth embodiment, there is provided a cosmetic composition carrier according to any one of the first embodiment to the eighth embodiment, wherein the latex foam may have a cell-in-cell structure including smaller cells in larger cells.

According to a tenth embodiment, there is provided a cosmetic composition carrier according to any one of the first embodiment to the ninth embodiment, wherein the latex foam may have antibacterial activity.

According to an eleventh embodiment, there is provided a cosmetic composition carrier according to any one of the first embodiment to the tenth embodiment, which may have at least one of the following characteristics:

i) a supportability of 13.5-14.8 g, as determined by measuring the amount of a cosmetic agent held in the carrier in a container after 15 g of the cosmetic composition is packed in the carrier and is allowed to stand for 4 weeks;

ii) a packability of 15-25 seconds, as determined by measuring the time required for packing 15 g of a cosmetic composition;

iii) a dischargeability of 0.3-0.5 g, as determined by measuring the pay-off amount of a cosmetic composition when carrying out pay-off of the cosmetic composition from the carrier with an applicator;

iv) an antibacterial activity of 99% or more, as determined by the test method according to KSK0693;

v) a height swelling ratio of 1-20% after supporting a cosmetic composition; and

vi) a diameter swelling ratio of 1-30% after supporting a cosmetic composition.

According to a twelfth embodiment, there is provided a cosmetic product which includes: the cosmetic composition carrier including a latex foam as defined in any one of the first embodiment to the eleventh embodiment; and a cosmetic composition supported in the carrier.

According to a thirteenth embodiment, there is provided a cosmetic product according to the twelfth embodiment, wherein the cosmetic composition may include an alcohol.

According to a fourteenth embodiment, there is provided a cosmetic product according to the twelfth embodiment or the thirteenth embodiment, which may further include an applicator with which the cosmetic composition is taken out of the latex foam.

According to a fifteenth embodiment, there is provided a cosmetic product according to any one of the twelfth embodiment or the fourteenth embodiment, wherein the cosmetic composition may have a viscosity of 3,000-30,000 cps.

According to a sixteenth embodiment, there is provided a cosmetic product according to any one of the twelfth embodiment or the fifteenth embodiment, wherein the cosmetic composition may have a formulation selected from the group consisting of a solution, emulsion, gel, cream and suspension.

According to a seventeenth embodiment, there is provided a cosmetic product according to any one of the twelfth embodiment or the sixteenth embodiment, wherein the cosmetic composition may be selected from the group consisting of a skin care cosmetic composition, makeup cosmetic composition, hair care cosmetic composition and a UV protecting agent.

1. A cosmetic composition carrier which comprises a latex foam having a density of 0.05-0.2 g/cm3.

2. The cosmetic composition carrier according to claim 1, wherein the latex foam comprises pores having an average pore size of 200-800 μm.

3. The cosmetic composition carrier according to claim 1, wherein the latex foam has a thickness of 1-50 mm.

4. The cosmetic composition carrier according to claim 1, wherein the latex foam has a hardness of 30-95 as determined by using a durometer before a cosmetic composition is supported therein.

5. The cosmetic composition carrier according to claim 1, wherein the latex is at least one selected from the group consisting of butadiene rubber (BR), styrene-butadiene rubber (SBR), natural rubber (NR), chloroprene rubber (CR), butyl rubber (isoprene-isobutylene rubber: IIR), isoprene rubber (IR), vulcanized ethylene-propylene rubber (EPR), polysulfide rubber, silicone rubber, fluororubber, urethane rubber, acrylic rubber, ethylene propylene diene monomer (EPDM) rubber, polyvinyl alcohol (PVA) and ethylene vinyl acetate (EVA).

6. The cosmetic composition carrier according to claim 1, wherein the latex foam comprises: styrene-butadiene rubber (SBR); and at least one of butyl rubber (isoprene-isobutylene rubber: IIR) and isoprene rubber (IR).

7. The cosmetic composition carrier according to claim 1, which is for use in taking and applying the cosmetic composition supported therein with an applicator.

8. The cosmetic composition carrier according to claim 7, wherein the latex foam has a coating film having closed cells on at least one of the bottom surface opposite to the surface in contact with an applicator and lateral surfaces.

9. The cosmetic composition carrier according to claim 1, wherein the latex foam has a cell-in-cell structure including smaller cells in larger cells.

10. The cosmetic composition carrier according to claim 1, wherein the latex foam has antibacterial activity.

11. The cosmetic composition carrier according to claim 1, which has at least one of the following characteristics:
i) a supportability of 13.5-14.8 g, as determined by measuring the amount of a cosmetic composition held in the carrier in a container after 15 g of the cosmetic composition is packed in the carrier and is allowed to stand for 4 weeks;
ii) a packability of 15-25 seconds, as determined by measuring the time required for packing 15 g of a cosmetic composition;
iii) a dischargeability of 0.3-0.5 g, as determined by measuring the pay-off amount of a cosmetic composition when carrying out pay-off of the cosmetic composition from the carrier with an applicator;
iv) an antibacterial activity of 99% or more, as determined by the test method according to KSK0693;
v) a height swelling ratio of 1-20% after supporting a cosmetic composition; and
vi) a diameter swelling ratio of 1-30% after supporting a cosmetic composition.

12. A cosmetic product which comprises:
the cosmetic composition carrier including a latex foam as defined in claim 1; and
a cosmetic composition supported in the carrier.

13. The cosmetic product according to claim 12, wherein the cosmetic composition comprises an alcohol.

14. The cosmetic product according to claim 12, which further comprises an applicator with which the cosmetic composition is taken out of the latex foam.

15. The cosmetic product according to claim 12, wherein the cosmetic composition has a viscosity of 3,000-30,000 cps.

16. The cosmetic product according to claim 12, wherein the cosmetic composition has a formulation selected from the group consisting of a solution, emulsion, gel, cream and suspension.

17. The cosmetic product according to claim 12, wherein the cosmetic composition is selected from the group consisting of a skin care cosmetic composition, makeup cosmetic composition, hair care cosmetic composition and a UV protecting agent.
